(19) United States
(12) Patent Application Publication
(10) Pub. No.: US 2009/0209857 A1
(43) Pub. Date: Aug. 20, 2009

(54) Method and system for identifying and quantifying particles in flow systems

(76) Inventors: Frank Secretain, Kingston (CA); Andrew Pollard, Kingston (CA); Christopher G. Ball, North Bay (CA); A.K. Mesbah Uddin, Charlotte, NC (US); Mohamed Ali, Richmond Hill (CA); Brian Milne, Kingston (CA); G. Andrew Y. Hamilton, Kingston (CA)

Correspondence Address: Ralph A. Dowell of DOWELL & DOWELL P.C., 2111 Eisenhower Ave, Suite 406, Alexandria, VA, 22314, US

(21) Appl. No.: 12/289,918

(22) Filed: Nov. 6, 2008

Related U.S. Application Data
(60) Provisional application No. 60/985,854, filed on Nov. 6, 2007.

(51) Int. Cl. A61B 8/00

(52) U.S. Cl. 600/437

(57) ABSTRACT

A method and system for quantifying particles in a flow system are provided.

### RELATED APPLICATIONS

This application claims the benefit of the filing date of U.S. Provisional Application No. 60/985,854, filed on 6 Nov. 2007, the contents of which are incorporated herein by reference in their entirety.

### FIELD OF THE INVENTION

The present invention provides a method and system for detecting and quantifying particles in flow systems such as biological and industrial flow systems. In one embodiment, the method and system of the present invention are useful in quantifying potential emboli introduced during surgery to the circulatory system thereby decreasing neurological dysfunction and mortality following the surgery.

### BACKGROUND OF THE INVENTION

Central nervous system (CNS) impairment ranging from stroke or coma to cognitive deficits is a major cause of morbidity after successful heart surgery (Roach et al. N. Engl. J Med. 1996 335:1857-1863). Cognitive decline, evident in as many as three quarters of patients at the time of hospital discharge, was present in 30-45% of patients after five years in one longitudinal study (Newman et al. N. Engl. J. Med. 2001 344(6):395-402). Neurocognitive dysfunction has a significant negative impact on hospital length of stay, productivity and the quality of life (Barbut et al. Ann. Thorac. Surg. 1997 63:998-1002; Newman et al. N. Engl. J. Med. 2001 344(6):395-402). Transcranial Doppler, retinal fluorescein angiography and brain histology studies indicate that cerebral micro-embolization from air or particulate matter or both constitute a major cause of perioperative neurological injury (Pugsley et al. Stroke 1994 25(7):1393-1399; Barbut et al. Ann. Thorac. Surg. 1997 63:998-1002; Blauth et al. J. Thorac. Cardiovasc. Surg. 1988 95:668-76; Moody et al. Ann. Thorac. 1990 28:477-86).

Open chamber cardiac procedures, such as valve repair or replacement, as well as surgical maneuvers, such as direct venting of cardiac chambers, create risk of air or solid embolism despite carefully performed, but unproven, procedures for ensuring removal. Solid particulate matter from the atherosclerosis of the ascending aorta or cardiac structures is the other major source of embolization and stroke.

Intra-cardiac procedures are accomplished by first arresting the heart with a potassium rich solution, while rerouting the blood around the heart and lungs by means of a cardiopulmonary bypass machine. The heart can then be opened, blood is removed from the heart cavities, and surgery is done to repair the heart. Upon completion of the intra-cardiac portion of the procedure, the heart is closed and de-airing is accomplished by progressively introducing blood into the heart while trapped air is permitted to escape by a variety of non-standardized techniques. These include: aspirating, agitating, compressing, venting, and altering patient position. The superiority and/or adequacy of a particular technique remain subjective.

Assessment and adequacy of the technique is generally based on visual 2-D images of echo-dense potential emboli in cardiac chambers obtained on transesophageal echocardiogram (TEE; Tingleff et al. Ann. Thorac. Surg. 1995 60:673-7), and not on objective quantification of the volume and size of air bubbles and particulate matter with potential for embolization to the brain. Further, surgical maneuvers remain unsophisticated and provide for incomplete removal of gaseous and solid emboli. Monitoring the removal process is typically accomplished utilizing medical ultrasound technology. However, gaseous and solid emboli are observed in cardiac chambers on TEE when the heart resumes its function. In addition, ultrasound technology commonly employed in current cardiac practice such as TEE (4-7 MHz) and phased-array linear transducer (6-15 MHz; Agilent Technologies, Inc., Santa Clara, Calif.) used most commonly for epicardial and epiaortic imaging only provide minimum resolution on the order of 100 μm. While this may be adequate to measure chamber size and aortic intimal thickness, it is crude in detection of potential emboli in the order of 40 μm or smaller.

Another common method of detection of air emboli is by transcranial Doppler (TCD). This method uses pulsed wave Doppler of 1-2 MHz focused on the middle cerebral artery (MCA). TCD uses fast Fourier transform methods and characteristic audible signals to distinguish emboli from artifacts (Ringelstein et al. 1998 29:725-9). However, TCD does not provide a visual image. Further, TCD detected emboli are operator and patient dependant as it is applied externally to the temple. Up to 30% of patients have a limited or inadequate window to insonate the MCA (Jarquin-Valdivia et al. J. Neuroimaging 2004 14(2):139-42). In addition, TCD detects air already in the brain and therefore is ineffective for the objective of detecting and removing air before it embolizes to the brain. Finally, TCD uses low frequency (˜2 MHz) that permits greater penetration at the expense of axial resolution (˜770 μm) with concomitant higher temporal resolution (˜78 μm at 1 m/s). To measure a 40 μm diameter air bubble, however, a higher transducer frequency is required.

Accordingly, there is a need for methods and systems for detecting and quantifying potential emboli such as air bubbles and solid particulates in flow systems such as the circulatory system to enable surgeons to best evaluate these potential emboli and to ensure the best neurological outcome following procedures such as open-heart surgery.

### SUMMARY OF THE INVENTION

According to one aspect there is provided a system for quantifying particles in a flow system. The system may include a means for non-invasively imaging a 2-dimensional or 3-dimensional region of a conduit of a flow system and a means for quantifying particles from the image. In one embodiment, the system may be used to quantify potential emboli in the circulatory system of an animal.

Another aspect provides a method for quantifying particles in a flow system. The method may include 2-dimensionally or 3-dimensionally imaging non-invasively a region of a conduit of the flow system and quantifying particles in the image. In one embodiment, the method is used to quantify potential emboli in the circulatory system of an animal.

Another aspect provides a module for use with an imaging system, the imaging system producing raw and/or preprocessed data relating to a flow system, wherein the module uses the raw and/or preprocessed data to detect and quantify particles within the flow system.

### BRIEF DESCRIPTION OF THE FIGURES

For a better understanding of the invention, and to show more clearly how it may be carried into effect, embodiments of the invention will be described below, by way of example, with reference to the accompanying drawings, wherein:

FIG. 1 is a diagram of a generalized embodiment including a detection device for non-invasively imaging a 2-dimensional or 3-dimensional region of a conduit of a flow system and a means for quantifying particles from the image.

FIGS. 2a and 2b are flow diagrams of the sequence of events occurring in an embodiment of the system and method, referred to herein as DETECTS™, used to quantify potential emboli in the circulatory system of an animal. FIG. 2a shows the entire sequence of events for quantifying emboli from start to stop while FIG. 2b shows additional detail of the sequence of events occurring between initialization and calibration of the algorithm and detection of the emboli.

FIGS. 3a through 3c provide exemplary images and graphs of data obtained using the DETECTS embodiment referred to above. FIG. 3a shows three 2-dimensional images of particles (air bubbles) detected ultrasonically by transesophageal echocardiogram (TEE) in the ascending aorta of a human following open heart surgery. FIG. 3b is a histogram showing size distribution of the air bubbles detected in the images of FIG. 3a. FIG. 3c is a line graph showing the volume percentage of air bubbles quantified as a function of time, for the images of FIG. 3a

FIG. 4 is a diagram showing how movements in the conduit wall (for example, an aorta wall) may be accounted for according to an embodiment of the method.

FIG. 5 is a schematic diagram of an experimental setup used to validate the DETECTS algorithm.

FIG. 6 is a plot showing results of an experiment performed to validate the DETECTS algorithm, in which optical data are compared with DETECTS data.

FIG. 7 is a plot comparing air bubble size determined using DETECTS to that measured optically.

### DETAILED DESCRIPTION OF EMBODIMENTS

One aspect provides a method and system for quantifying particles in a conduit of any flow system.

The term “flow system”, as used herein, includes any open or closed single or multiphase system with a gas, liquid and/or solid flow moving through or into and out of the system. One example of a flow system is one having a liquid carrier, in which gas, liquid, and/or solid particles may be present. Another example of a flow system is one having a gas carrier, in which liquid and/or solid particles may be present. Flow systems may include, but are not limited to, biological systems such as the circulatory system of an animal, industrial pipelines and ducting systems.

The term “animal”, as used herein, includes mammals, and in particular humans, as well as birds and reptiles.

The term, “particle”, as used herein, refers to a quantity of matter separated from its surroundings by a phase boundary. The term, “phase”, as used herein, refers to a quantity of matter that is homogeneous in physical structure within a boundary. Homogeneity in physical structure means that the matter is all solid, or all liquid, or all vapour (or equivalently all gas). When more than one phase is present, the phases are separated by a phase boundary. Note that gases, such as nitrogen and oxygen, can be mixed in any proportion to form a single phase, while certain liquids such as oil and water form two liquid phases. The method and system described herein may be used to detect and quantify particles in a flow system by detecting such a phase boundary between the particles and their surroundings.

Particles may be introduced into a flow system intentionally (e.g., for delivery within a system) or unintentionally (e.g., as a contaminant, a by-product of a process, etc.). In either case, such particles may be detected and quantified as described herein.

Examples of a particle include a gas-filled bubble, a solid particulate, and a liquid droplet. Examples of particles in a biological system such as the circulatory system of an animal include, but are not limited to, thrombi (including, for example, blood clots and bile thrombi), air bubbles, fatty deposits, tissue, bacteria, and any other embolus that may be carried in the bloodstream.

The term “embolus”, “emboli” or “potential emboli”, as used herein, includes any such particle in a biological flow system that can or does lodge in a vessel or conduit thereby causing an embolism.

Detection of a particle as described herein may include resolving a phase boundary of 10 μm or smaller, for example 5 to 10 μm. Detection of smaller particles may be carried out, and is limited only by the limitations inherent in the imaging technique used. The resolution of an imaging technique may be in turn be limited by currently available technology, but may be expected to improve as technology (e.g., manufacturing capability, etc.) improves. For example, where the imaging technique is ultrasound, the resolution may be limited by the ultrasound transducers, but the resolution of the technique may be improved with advancements in transducer design and fabrication. It will be appreciated that the method and system described herein are independent of the imaging technology used. That is, the method and system may be used with any current or future imaging technology from which phase boundary information may be derived.

The term “quantifying”, “quantify” or “quantifies”, as used herein, includes detection of particles and/or determination of one or more of the type, size distribution and number of particles in real time in the flow system. Thus, for any given flow system, the method and system of the present invention can be used to determine the type of particle(s), the size(s) of the particle(s), and the number of each type of particle. However, the invention is not limited to such determinations.

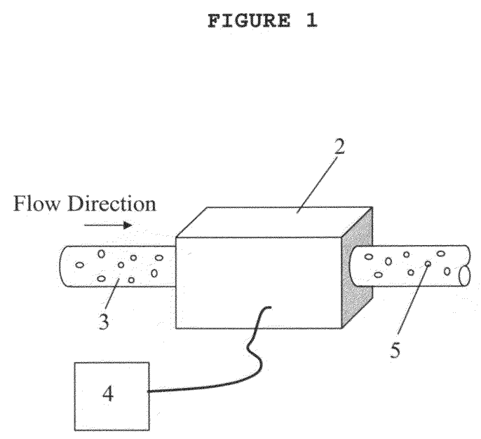

FIG. 1 provides a diagram of an embodiment of the system in its simplest form. As shown therein, the system comprises a detection device 2 which uses a non-invasive technique to generate a 2-dimensional image of the flow in a region of the conduit 3 or a 3-dimensional image of the flow in a region of the conduit 3 as a function of time. Exemplary detection devices that may be used include, but are not limited to, imaging technologies such as those using acoustics (e.g., ultrasound), optics, x-rays, or magnetic resonance. The image created by the detection device is a volumetric representation of the flow and any particles 5 therein. Once captured, the image is then analyzed via a means 4 for quantifying particles from the image. This means 4 comprises an algorithm capable of quantifying particles of the captured image and an output device for display of data.

According to the method, a 2-dimensional or 3-dimensional image of a region of a conduit of a flow system is noninvasively measured. Particles in the image are then quantified from either raw data of the image or processing of the image.

The method and system are particularly useful in quantifying particles such as potential emboli introduced during surgery to the circulatory system thereby decreasing neurological dysfunction, morbidity and mortality following the surgery. Neurological dysfunction is one of the main negative outcomes suffered following open heart surgery. Heart valve replacement surgery alone carries a 5% mortality risk because of the possibility of stroke caused by air bubbles or solid emboli entering the bloodstream. Despite efforts to remove air (or other emboli) from the circulatory system, air bubbles are frequently observed with the transesophageal echocardiography (TEE) once the patient has been separated from cardiopulmonary bypass machine. The ultimate goal is to eliminate all negative neurological outcomes caused by air emboli following cardiac surgery. Current air removal techniques are already employed in the operating room but the effectiveness of these techniques is still unknown.

Accordingly, a further detailed description of the system and method is provided in relationship to its use in quantifying potential emboli of the circulatory system. However, as will be understood by the skilled artisan upon reading this disclosure, such detailed description is non-limiting, and the system and method described herein may be easily adapted to be used with other flow systems, including other biological flow systems and non-biological flow systems. For example, the system and method may be applied generally to the field of mechanical engineering, where other imaging techniques based on, e.g, electromagnetic, acoustic, etc., technologies, may be used. Examples include but are not limited to the transport of solid particles in a gaseous carrier (such as flocculents in air) or a liquid carrier (such as wood chips in water), or liquid particles (e.g., droplets) in a gaseous or liquid carrier, such as oil in water. Such flow systems may be present in, for example, pulp and paper facilities, chemical industries, particle delivery systems such as coal delivery, fuel injection systems in vehicles, and drug delivery systems such as pulmonary drug delivery systems.

For use in quantifying emboli, detection may be carried out using a non-invasive imaging technique such as ultrasonic imaging. Exemplary ultrasonic imaging techniques include, but are not limited to phased array crystal such as, but not limited to, TEE, step down segmental linear crystal such as, but not limited to, a footprint transducer, and dynamic receiving focusing. Although the operating frequency is limited to the manufacturability of the transducer, most useful because of the short scan area (average aorta ranges from 2.5-4.0 cm) is an imaging technique capable of operating at higher frequencies, for examples, greater than 50 MHz. Operation at higher frequencies significantly improves axial resolution. Also useful are transducers with a thickness approximately half the axial resolution for optimal performance, which for this application is on the order of 50 μm. A benefit of these imaging techniques as compared to, for example, TCD technology, is that they cannot be overloaded by large quantities of emboli.

To provide real-time, online data during open-heart surgery, the ultrasonic device may be designed for attachment to the aorta, to a cardio-pulmonary bypass machine, or intermediate/associated conduits.

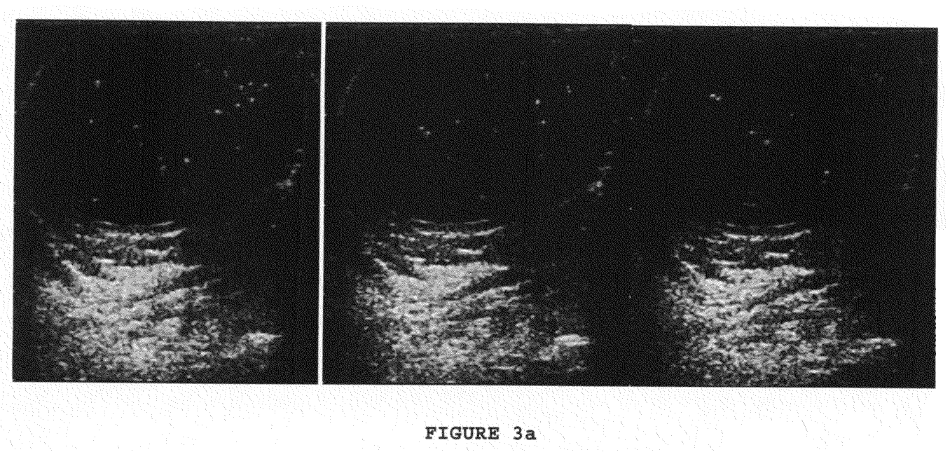

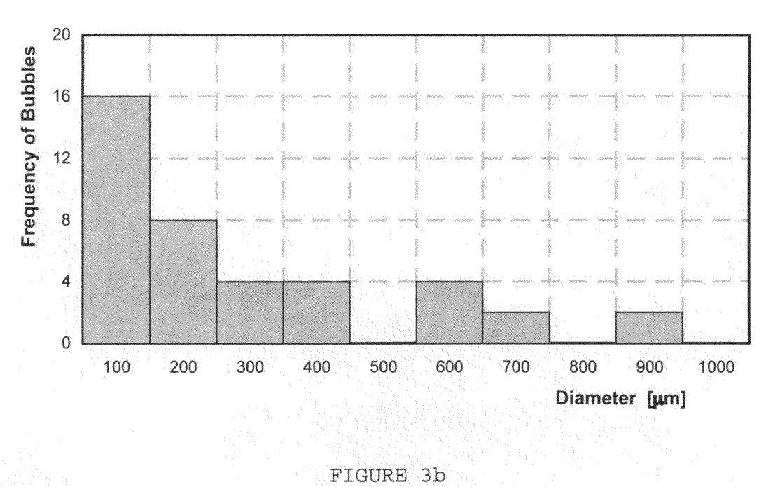

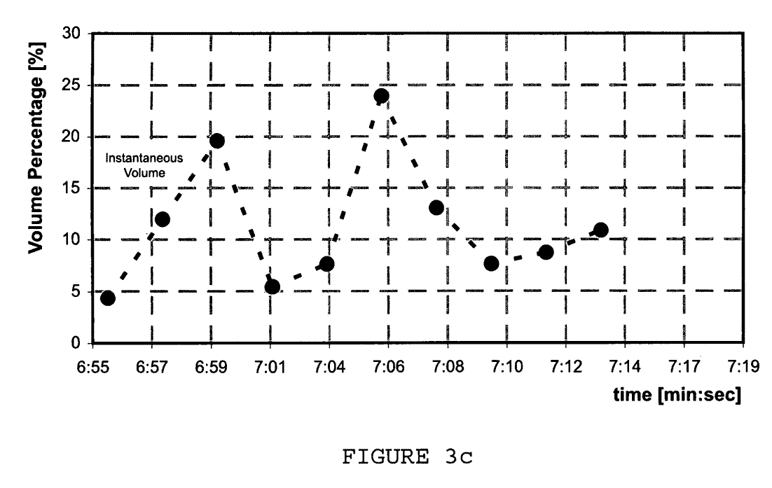

Exemplary 2-dimensional images of the aorta obtained using TEE are depicted in FIG. 3a.

This embodiment of the system and method detects particles using minimum and maximum intensity threshold values, and then verifies particle shapes (e.g., spherical vs. spheroid, in the case of gas bubbles) by comparison with data from the literature (see for example Clift et al., Bubbles, Drops and Particles, Academic Press (1978)). For each image frame, total bubble area is recorded and interpreted in order to provide the surgical team or other users of the system with information about the total air volume entering systemic circulation. Shadowgraphy techniques are used for direct verification of the proposed ultrasound detection and algorithmic interpretation of total air bubbles. Access to the pre-processed data from the ultrasonic transducer also enables air bubbles to be differentiated from solid emboli and is subjected to the same analysis as for air bubbles. Thus, for each image frame, total amounts of air and solid emboli are calculated and presented to the surgical team.

An exemplary algorithm for use in an embodiment for quantifying emboli of the circulatory system of an animal is referred to herein as DETECTS™ (Detection of Emboli using Trans-esophageal Echocardiography for Counting, Total volume, and Size estimation). DETECTS interprets the two-dimensional images obtained from the imaging technique, and analyses them for the presence and quantities of emboli. In one embodiment, DETECTS is an add-on for existing ultra-sound hardware, for sensing emboli passing through the plane of the image and providing data describing, e.g., the count, volume total, and size distribution of the emboli. Such an embodiment minimizes post-operative mortality and neurological disfunction, and provides a quantitative tool for the development of surgical standards for emboli measurement during procedures such as open heart surgery. DETECTS may of course be adapted for use in quantifying particles in other biological and non-biological flow systems.

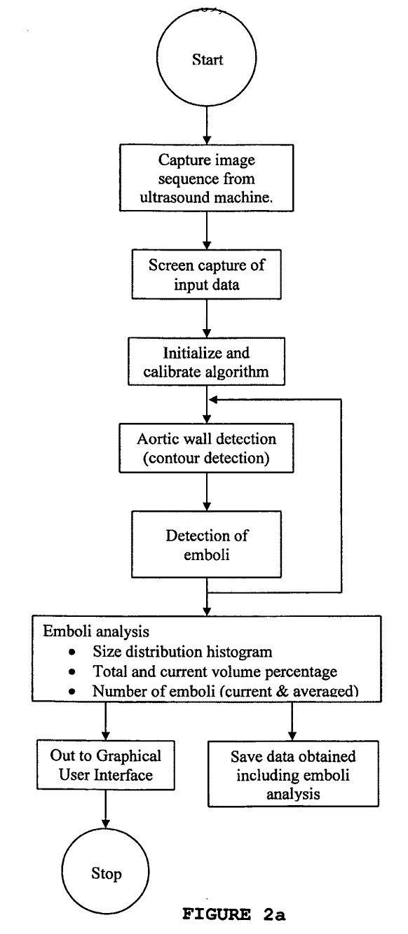

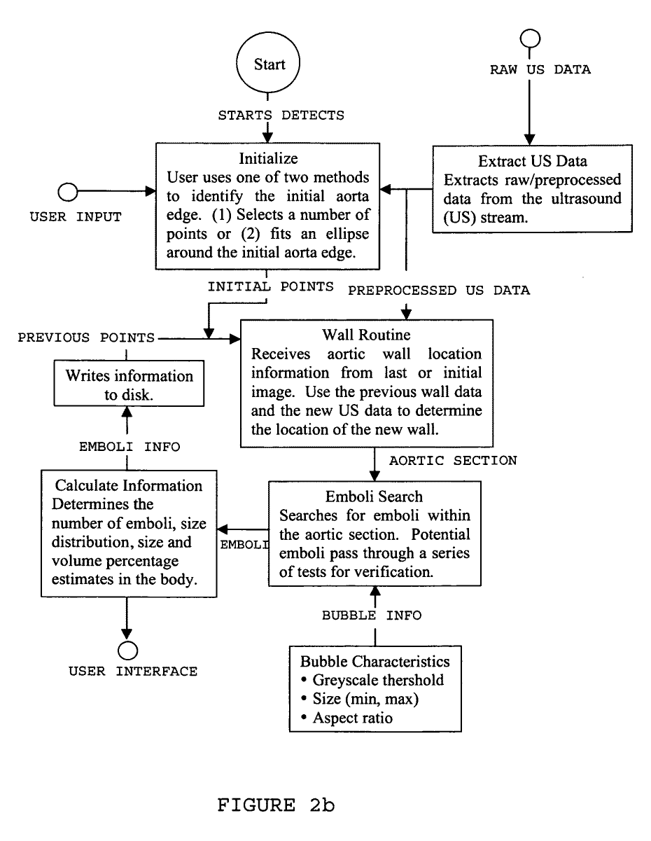

A flow diagram of the sequence of events of DETECTS, when used in the three-phase flow system of the circulatory system of an animal, is depicted in FIGS. 2a and 2b. FIG. 2a is a flow diagram showing the entire sequence of events from start to stop of emboli detection while FIG. 2b provides a more detailed flow chart of the sequence of events from initialization and calibration of the exemplary algorithm DETECTS to emboli detection within the human body using ultrasound technology. A more detailed description of the sequence of events occurring in the exemplary algorithm DETECTS™ is set forth in Example 1. However, steps and details may change depending on the application and detection method.

DETECTS instantaneously quantifies the total amount of air per frame (or as percentage), the total number of bubbles per frame, and the size distribution of bubbles in frame. DETECTS then averages the total amount of air over averaged time (or as percentage), the total number of bubbles over averaged time, and the averaged size distribution of bubbles over averaged time.

To determine the volumetric percentage of, for example, air bubbles, edge detection on the initial image is performed and the inner wall of the aorta or ventricle is detected.

Various techniques may be used to detect the inner wall such as, but not limited to, the RANSAC method and the least squares method. The least squares method fits the data to a given curve (the type of curve/function is chosen by the user). For DETECTS, the curve may be an ellipse or a more complicated function than an ellipse. The curve is fit to the set of data points by minimizing the residual error, where the residual error is defined as the sum of the squares between each point and the function. The distance between the point and the function may be determined using two either the vertical offset or the perpendicular offset of each point relative to the function.

The RANSAC method (see Fischler et al., Random Sample Consensus: A Paradigm for Model Fitting with Applications to Image Analysis and Automated Cartography. Communications of the ACM, 1981 24:381-395) picks a random set of points, and then tries to fit an equation of a circle (or another shape) to it while rejecting and retrieving more data to be used. After a certain threshold of accuracy has been reached, the program terminates and restarts with a new set of points excluding those just fitted. This repeats over and over again until another minimum threshold is reached with the remaining points and all the objects in the image have been recognized. Alternatively, a point on the inner wall is found and then followed around the aorta. The initial point may be found several ways, for example, by moving horizontally or vertically and stopping at the second interface. This method may be performed from several different angles and starting locations to be positive on the correct identification of the inner aorta.

After the inner wall of the aorta has been resolved, this embodiment of DETECTS returns to the initial image and uses a gray scale threshold to determine the percentage of bubbles in the flow. A gray scale image represents the image in shades of gray from black to white. A gray scale threshold is a value in which if a shade (or value) of a certain pixel is above, it passes the threshold, and if below, does not pass the threshold. As no image recognition is required, this greatly saves on computational time. Further, it is more precise than trying to fit a circle to a bubble that might not be exactly spherical. In addition, other information including, but not limited to size distribution, mean size and errors can be determined after the bubbles have been resolved.

It will be appreciated that a gray scale threshold is one example of a thresholding function that may used. Any suitable mathematical function may be used for thresholding to fit the detected particle to the function, such as, for example, but not limited to, a Gaussian function or a polynomial function, or combinations of functions.

Air bubbles are distinguished from solid particles by differences in their acoustic reflectances. For example, gaseous reflectance is on the order of about 99%, while solid reflectance is on the order of about 1% transmitted after pre-processing from the ultrasound transducer. This number includes time gain compensation, selective enhancement, logarithmic compression fill-interpolation, and edge-enhancement (see for example, Hedrik et al. Ultrasound physics and Instrumentation, Mosby Inc. St. Louis (1995) pages 208-237). Furthermore, accurate volumetric measurements of air and particulates that enter the circulatory system is possible because of the frame rate employed in lateral imaging, which is parallel to the transverse plane of the aorta.

This embodiment of the system is particularly useful in emboli detection because of the speed of the DETECTS algorithm. Its speed enables real-time online emboli measurement during surgery. Further, use of this embodiment of the system provides an accurate real-time measure of the surgeon's emboli removal technique.

Exemplary data of particles quantified via analysis of the 2-dimensional images of the ascending aorta of FIG. 3a by DETECTS are shown in FIGS. 3b and 3c. As shown, analysis of the ultrasonic image via DETECTS gives accurate values on the number and size distributions of air bubbles passing through the ascending aorta (FIG. 3b). When combined with real time data from the TEE, the volume of air and volume percentage in the circulatory system was determined (FIG. 3c).

Using embodiments as described herein, the effectiveness of current emboli removal techniques can be assessed thereby greatly improving neurological outcome post cardiac surgery (see, e.g., Example 2). Accordingly, the system and method extends the functionality of existing ultrasound systems to influence and improve the patient outcome of open-heart surgery. The system and method accurately measures flow rates of particles while simultaneously providing quantitative data on parameters including, but not limited to, particle size, volume, and count. This on-line, real-time information gives physicians the ability to objectively gauge emboli-content status of a patient's circulatory system as it is being weaned from the pulmonary bypass system at the conclusion of open-heart surgery, thus permitting specification of standards for air and solid particle removal in the closing stages of cardiac surgery.

As noted above, magnetic resonance imaging (MRI) is another example of an imaging method that can be used with the invention to detect and quantify particles in a conduit of a flow system. MRI is a non-invasive technique using nuclear magnetic resonance to render images within specified volume. MRI can produce much higher resolution images than the ultrasound imaging. For example, a conduit maybe placed within a MRI machine (longitudinal to the primary axis of the MRI machine) and a flow medium (e.g., gas, liquid, etc.) pumped down the conduit. MRI specifically detects the resonance of hydrogen atoms in the media/flow; and particles within the flow having less or no hydrogen than the flow media (such as, for example, air bubbles within a water flow in an industrial process) may be detected and classified as voids within the flow. Thus, air bubbles, for example, may be detected in the flow and the bubble statistics maybe calculated in the conduit as done in the examples described herein using ultrasound data.

Another example of an application, in a biological system, to which the method and system described herein may be applied is in detection and evaluation of the patent foramen ovale (PFO). In the fetal heart, the foramen ovale allows communication between the right atrium (RA) and left atrium (LA). The PFO is a remnant of the fetal circulation characterized by incomplete closure of the foramen ovale after birth, which may result in circulatory problems in children and adults. For example, presence of the PFO is a common finding with an incidence of up to 35% in autopsy studies of adults.¹ PFO has been associated with cryptogenic stroke, hypoxemia, decompression sickness, and migraine headache. There is generally a positive correlation with the size of the PFO and the association with these adverse events.² The principle approach to PFO detection is echocardiography, with transesophageal echocardiography(TEE) generally considered more sensitive than transthoracic echocardiography (TTE). Various echocardiographic techniques to measure the size of a PFO have been studied. These include two-dimensional imaging, transmitral Doppler, but semi-quantitative contrast studies have been widely used as to quantify the size of a PFO.^{3, 4}. A contrast study consists of the injection of contrast agent, usually agitated saline, in the RA by way of a vein and then observing the passage of microbubbles into the LA and counting the maximum number of bubbles in a single echo frame. This semi-quantitative approach, although widely used, has been shown to be less accurate than other techniques to quantify size.⁵

TEE is routinely used during coronary artery bypass graft (CABG) surgery to monitor ventricular and valvular function. A PFO is a common incidental finding during the comprehensive intraoperative TEE examination. Controversy exists regarding whether closure of the incidentally found PFO should be done at the time of CABG.⁶

Applying the method and system described herein (e.g., DETECTS) would automate contrast microbubble quantification as well as give total numbers of microbubbles that are observed to pass through the PFO. This would avoid having to rely on a measure of the maximum number of bubbles in a single frame, as is done using TEE.

As the size of PFO is generally associated with adverse outcomes, the ability to easily and accurately quantify the size of a PFO, using a method and system as described herein, would be valuable both as a research tool and as a screening tool for patient who may be at risk for adverse outcomes. It may also help in the determination of a PFO that should indeed be closed at the time of cardiac surgery.

The contents of all references, pending patent applications, and published patents cited throughout this application are hereby expressly incorporated by reference.

The following nonlimiting examples are provided to further illustrate embodiments of the invention.

### EXAMPLE 1

### Description of Events in DETECTS™ Flow Chart (FIG. 2B)

This example provides a more detailed description of each of the events occurring in FIG. 2(b), DETECTS flow chart. This flow chart and the following detailed events are directed towards quantifying emboli within the human body using ultrasound technology.

In the first event of “Extract US (ultrasound) Data” shown in FIG. 2b, raw or preprocessed ultrasound data are extracted from the ultrasound stream. Preprocessing for an ultrasound image may include one or more of: time gain compensation, selective enhancement, logarithmic compression, fill-in interpolation, edge enhancement and write zoom. Preprocessing essentially creates an image that is recognizable by humans. DETECTS can extract the raw or preprocessed data from the ultrasound machine, where if the raw data are extracted all the preprocessing would be done internally by DETECTS. Advantages of dealing with the raw data are that the estimations of the preprocessing step are known. This creates more accurate values for the data analyses (e.g., volume percentage, histograms of size distribution, etc.). The data are extracted before post processing (image display) where additional enhancements and averaging is done to the image.

In the following event of “Initialize”, the initial aortic wall boundary is determined. When activating DETECTS™ an ultrasound image is frozen and the user is prompted to select a wall value. The point selection may be performed by various methods. Examples include, but are not limited to, selecting a number of points and fitting an ellipse to the rough aorta edge. The user's points will be used to fit a contour to the edge of the aorta in the following event “Wall Routine”.

Streaming ultrasound images in preprocessed format are input while wall boundary, confidence level on selection, and percentage error are output.

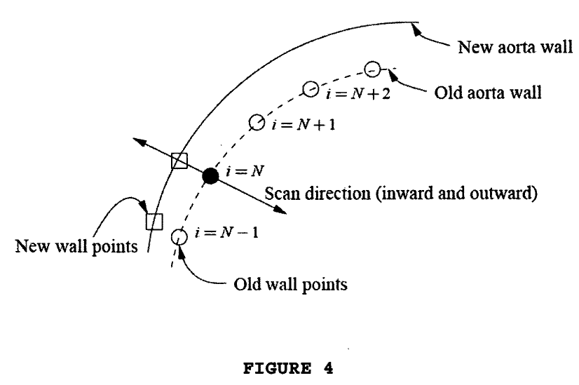

In the following event “Wall routine”, the boundary of the aorta is updated to account for movements in the aorta since the last ultrasound scan. Each point on the aortic wall scans in a normal direction to the previous aortic wall surface. The scan looks inward and outward for characteristics to identify the new location of the aortic wall (see FIG. 4). Characteristics include, but are not limited to, a sharp increase in grayscale shade and a grayscale gradient increase. The new location is compared to the previous aortic wall location for purpose of error checking. The checks preformed include, but are not limited to, the global percentage increase of the aortic area threshold and selected points completing the closed contour.

Initial or previous wall boundary is input while current wall boundary and error are output.

In the following event of “Emboli Search”, the input aortic image for emboli is scanned. Potential emboli are detected with an increase in grayscale threshold and then checked versus a series of criteria. This process includes, but is not limited to, scanning horizontally within the aortic section until a threshold value is met and scanning the potential emboli with the algorithm checking for maximum aspect ratio and maximum and minimum bubble diameter. If these criteria are met the emboli's position and size are saved in an array and sent to the event “Calculate Information”.

The event “Bubble Characteristics” is used for the grayscale threshold cutoffs and bubble parameters.

Ultrasound images and aortic wall values are input while threshold value and bubble characteristics as well as bubble locations are output.

In the event “Calculate Information” emboli data is analyzed using standard statistical analysis. This includes, but is not limited to, visual detection of bubbles displayed on screen, emboli area percentage including current, averaged (x frames) and total in blood, emboli size distribution including current, averaged (x frames) and total, and emboli volume versus time including current, averaged (x frames) and total.

Bubble locations are input while emboli/blood area percentage, emboli/blood average area percentage versus time, number of emboli circled and layered on the ultrasound image, and emboli distribution as a histogram are output. The graphical user interface (GUI) and selection of which figures to display and where on the screen are options.

### EXAMPLE 2

### Validation of DETECTS™

### 1. Introduction

The DETECTS algorithm was designed to identify and measure potential emboli present during and post cardiopulmonary bypass. DETECTS software uses existing Acuson/Antares™ (Siemens AG) ultrasound technology to quantify the amount of potential emboli in consecutive images from the ultrasound machine. The DETECTS algorithm is the first software package that can reliably detect multiple potential emboli per TEE image. It can accurately measure the number and size of potential emboli, which provides a means to quantitatively evaluate the effectiveness of currently existing emboli removal techniques. Better emboli removal techniques lead to improved neurological outcomes post cardiac surgery.

DETECTS measures the reflected acoustic signal from air bubbles, to produce a two dimensional slice of the measurement volume. The intensity of the acoustic signal is related to the size of the air bubbles being measured. The intensity is also a function of the input acoustic frequency, power, shape of the bubble and number of bubbles in the vicinity (reflections and absorptions of other bubbles). This in vitro experiment was performed to determine the relationship between the true size of the bubble, measured optically, and the size reported by the DETECTS algorithm. These measurements will allow for the acoustic signal (with knowledge of the incident signal), to be related to the true size of the bubble.

### 2. Experimental Methodology

### 2.1 Experimental Apparatus

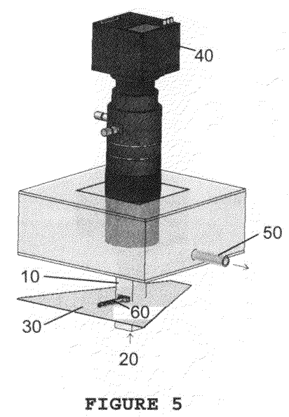

The experimental apparatus is shown in FIG. 5. A bubbler, not shown, included a pneumatic cylinder, piping, valves, and connections to produce bubbles within a rise chamber 10. Air was delivered using the pneumatic cylinder through one of four inputs into the rise chamber. The air bubbles were produced at the ends of glass tubes that were connected to one of the four air inputs. The glass tube ends were created by pulling glass pipettes under a flame to produce outlet diameters on the order of hundreds of microns.

The rise chamber 10 was constructed using clear Plexiglas™ fastened using aquarium tank glue and screws. A laminate sheet was used on the side of the rise chamber to provide access to the ultrasonic transducer. Air bubbles introduced in the bottom of the rise chamber (see arrow, 20) rise under the influence of gravity and travel through the measurement plane of the optical and DETECTS systems. The air bubbles were then allowed to escape through a pipe 50 at the top the rise chamber.

### 2.2 Optical Bubble Detection System

An optical measurement plane was created using two perpendicular 250 Watt halogen lamps and a narrow slit in the rise chamber. Light entered the rise chamber through the narrow slit. This produced a light sheet 30 inside the middle of the rise chamber. The rest of the validation apparatus was covered with black cloth to only allow light in through the optical slit. A High Definition Sony Handycam™ video camera 40 was oriented to view down into the rise chamber, normal to the light sheet. As the air bubbles rose through the rise chamber they passed through the light sheet and reflected light, which was recorded by the video camera.

### 2.3 DETECTS™ System

An ultrasonic footprint transducer 60 (VF13-5SP, Siemens AG) was orientated in line with the optical slit on the side of the rise chamber. The laminate sheet separated the transducer face from the liquid inside the rise chamber. The Antares ultrasound system was used to ultrasonically detect the air bubbles in the fluid as they passed through the transducer view plane. The DETECTS algorithm was used to analyze the detected bubbles.

### 2.4 Experimental Procedure

The general procedure is summarized below:
1. Turn on all systems, including DETECTS™, video camera and halogen lights.
2. Set up DETECTS, which includes:
   a. Zoom out to encompass the maximum range of the rise chamber cross section.
   b. Focus the ultrasound (US) beam to the center of the rise chamber.
   c. Adjust the gain and mechanical index (MI).
   d. Start the DETECTS™ algorithm.
   e. Choose a region of interest (ROI) within the transducer view range.
3. Set up the optical system by zooming the camera's view to approximately the same viewing area of the ultrasound transducer (the exact offset is determined post experiment).
4. Open the main valve to allow air to be forced from the pneumatic cylinder to one of the inputs into the rise chamber.
5. Start recording with both DETECTS and the video camera at approximately the same time (the exact time lag is determined post experiment).
6. Manually push down the pneumatic cylinder to produce the bubbles into the rise chamber. Turn on and off valves into the rise chamber to adjust the bubble radii.
7. Stop recording of the DETECTS and video camera at approximately the same time.
8. Transfer the DETECTS and video data to an external computer and shut down all systems.

The raw data from the DETECTS algorithm was analyzed directly without any modification. The optical data was retrieved from the video camera and converted to a series of frames. A computer program was created to automatically process the optical frames. This included thresholding the images and then measuring the number and size of the bubbles in the frame. The data from each of the measurement techniques was then compared.

### 3. Results

The data from both of the measuring techniques were compared based on number of bubbles detected, the location of bubbles, and the bubble radii reported. Qualitative and quantitative analysis gave good correlation between results from the DETECTS algorithm compared to optical measurements. The data show that individual air bubbles were distinguished from each other and relative sizes of the bubbles were determined.

The shape and intensity of the reflected acoustic intensity was determined to uniquely characterize the true bubble radii. The DETECTS algorithm was least affected by mechanical index and gain of the ultrasound machine. The detection of bubbles either optically or using DETECTS was most affected by the grayscale threshold parameter.

### 3.1 Optical Imaging Results

The computer program received the threshold images and automatically determined the number of bubbles, and location and radii of the bubbles in each frame.

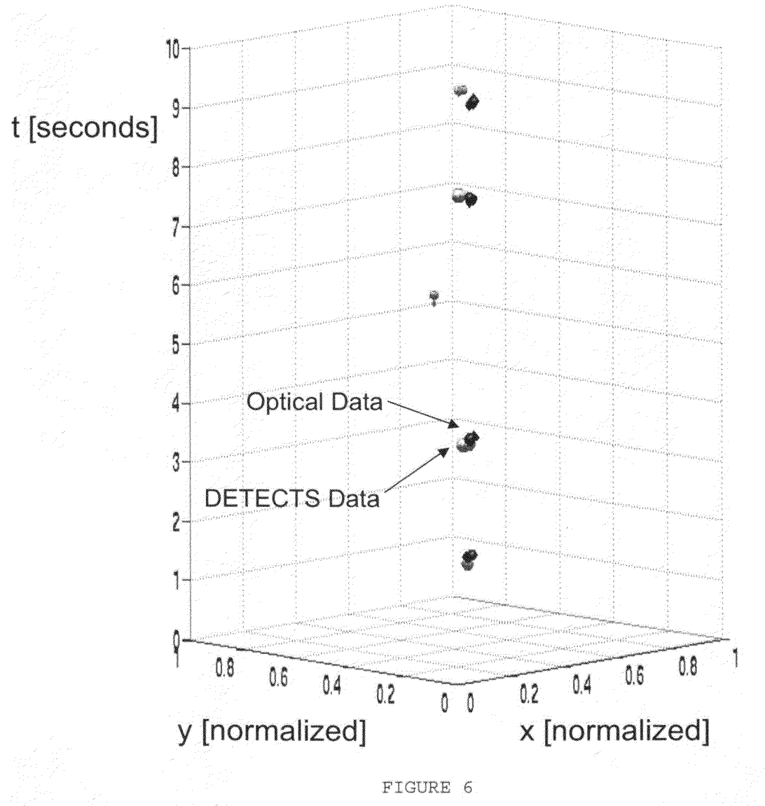

The optical data were plotted (FIG. 6) volumetrically as a function of time, i.e., each time slice represents one frame of the measurement plane recorded using the video camera. As seen in FIG. 6 the bubbles and relative sizes are easily distinguishable from each other.

### 3.2 DETECTS Imaging Results

The DETECTS algorithm detected and counted bubbles by applying a grayscale threshold on the original acoustic image and counting the resultant bubbles. The largest influence on the DETECTS detection was the grayscale threshold. The DETECTS data are plotted volumetrically as a function of time over the same time period as the optical data in FIG. 6.

### 3.3 Comparison of Optical and DETECTS Results

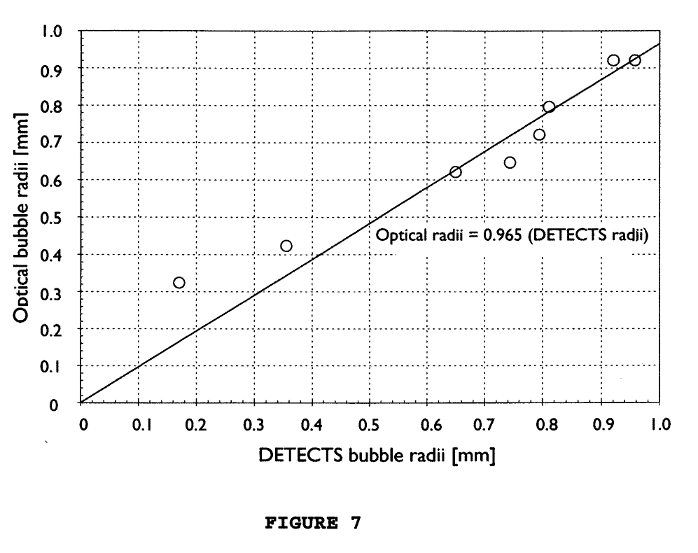

The DETECTS and optical data were compared to determine the relationship between calculated bubble size from the DETECTS algorithm and actual bubble size. As seen in FIG. 7, the general trend is observed to be linear between the true bubble size and the bubble size recorded by DETECTS. FIG. 7 clearly shows that the acoustic images from the bubbles correlate with the actual bubble sizes measured optically.

### 4. Conclusions

From the results it may be concluded that detection of emboli using trans-esophageal echocardiography (or any ultrasonic transducer) for counting, total volume, and size estimation (i.e., DETECTS) provides real time air emboli information. The information may be used by a cardiac surgery team during de-airing of the heart, to quantitatively evaluate the effectiveness of current emboli removal techniques, which in turn will lead to improved outcomes post cardiac surgery.

### REFERENCES

1. Thompson T, Evans W. Paradoxical embolism. Q J Med. 1930; 23:135-50.
2. Kerut E K et al. Patent foramen ovale: A review of associated conditions and the impact of physiological size. J Am Coll Cardiol 2001; 38:613-23.
3. Schuchlenz H W. Transesophageal echocardiography for quantifying size of patent foramen ovale in patients with cryptogenic cerebrovascular events. Stroke 2002; 33:293-6.
4. Kerr A J et al. Transmitral doppler: A new transthoracic contrast method for patent foramen ovale detection and quantification. J Am Coll Cardiol 2000; 36:1959-66.
5. Schuchlenz H W et al. The association between the diameter of a patent foramen ovale and the risk of embolic cerebrovascular events. Am J Med 2000; 109:456-62.
6. Sukernik M R, Bennet-Guerrero E. The incidental finding of a patent foramen ovale during cardiac surgery: should it always be repaired? A core review. Anesth Analg 2007; 105:602-10.

What is claimed is:

1. A system for quantifying particles in a flow system, comprising:
a detection device for imaging a 2-dimensional or 3-dimensional region of a conduit of the flow system; and
a means for quantifying particles from the image.

2. The system of claim 1, wherein the detection device uses an imaging technology selected from acoustic, optical, x-ray and magnetic resonance imaging.

3. The system of claim 1, wherein the detection device uses acoustic imaging technology.

4. The system of claim 3, wherein the acoustic detection device comprises a transesophageal echocardiograph transducer or a footprint transducer.

5. The system of claim 1, wherein the detection device is designed to attach to an aorta of an animal.

6. The system of claim 5, wherein the animal is a human.

7. The system of claim 1, wherein the means for quantifying particles comprises an algorithm capable of:
extracting image data;
detecting particles according to a mathematical thresholding function; and
outputting data relating to particles in the image.

8. The system of claim 7, wherein the algorithm is DETECTS™.

9. The system of claim 7, wherein the means quantifies particles from raw data of the image.

10. The system of claim 7, wherein the means quantifies particles by processing the image.

11. A method for non-invasively quantifying particles in a flow system, comprising:
2-dimensionally or 3-dimensionally imaging non-invasively a region of a conduit of the flow system; and
quantifying particles in the image.

12. The method of claim 11, wherein the 2-dimensional or 3-dimensional image is generated using an imaging technology selected from acoustic, optical, x-ray, and magnetic resonance imaging.

13. The method of claim 11, wherein the 2-dimensional or 3-dimensional image is generated via an acoustic imaging technology.

14. The method of claim 13, wherein the acoustic technology comprises a transesophageal echocardiograph transducer or a footprint transducer.

15. The method of claim 11, wherein the flow system is the circulatory system of an animal and the conduit is the aorta.

16. The method of claim 15, wherein the particles quantified are potential emboli.

17. The method of claim 15, wherein the animal is a human.

18. The method of claim 17, wherein the human is undergoing a surgical procedure.

19. The method of claim 11, wherein quantifying particles in the image comprises:
extracting image data;
detecting particles according to a mathematical thresholding function; and
outputting data relating to particles in the image

20. The method of claim 19, wherein quantifying includes a DETECTS™ algorithm.

21. The method of claim 19, comprising quantifying particles from raw data of the image.

22. The method of claim 19, comprising quantifying particles by processing the image.
